(19) United States
(12) Patent Application Publication
(10) Pub. No.: US 2022/0168459 A1
(43) Pub. Date: Jun. 2, 2022

(54) Germicidal Lighting With Controlled Ventilation

(71) Applicant: AURA BLUE LLC, Sacramento, CA (US)

(72) Inventors: Biane Goettle, Golden River, CA (US); Yan Zhou, Sacramento, CA (US); James Albert Gavney, JR., Sunnyvale, CA (US)

(73) Assignee: AURA BLUE LLC, Sacramento, CA (US)

(21) Appl. No.: 17/300,570

(22) Filed: Aug. 19, 2021

Related U.S. Application Data
(60) Provisional application No. 63/103,743, filed on Aug. 20, 2020.

(51) Int. Cl. A61L 9/20

(52) U.S. Cl. CPC A61L 9/20; A61L 2209/12

(57) ABSTRACT

A germicidal light fixture includes a disinfecting chambers or germicidal camber with a UV or UVC light engine therein for treating air. The disinfecting chamber is in communication with ventilation that includes for examples fans the draws untreated air into the disinfecting chamber and pushes treated and sanitized air out of the germicidal chamber. To control residency times that volumes of air spend within the disinfecting chamber, stationary baffles, ribbons, tabs or other structures or structures protrude from interior walls of the disinfecting chamber.

### RELATED APPLICATION

This application also claims priority under 35 U.S.C. § 119(e) from the U.S. provisional patent application Ser. No. 63/103,743, filed on Aug. 20, 2020 and titled “GROW-LIGHT SYSTEMS WITH GERMICIDAL LIGHTING” the contents of which is hereby incorporated by reference.

### FIELD OF THE INVENTION

The invention relates to germicidal lighting systems. More particularly, the present invention relates to germicidal lighting systems with controlled ventilation.

### BACKGROUND OF THE INVENTION

Germicidal light fixtures produce some amount of short-wave ultraviolet (UVC) that can break of disrupt DNA base pairing, causing formation of pyrimidine dimers, and leading inactivation of bacteria, viruses, and protozoa. Germicidal light fixtures are used in water disinfecting systems. Germicidal light fixtures can include low-pressure lamps, high-pressure lamps, LEDs or combination thereof.

Germicidal light fixtures can be used to sterilize workplaces tools and the like. One of the problems with Germicidal light fixtures is that the UVC light that they emit is harmful to humans and can cause burning of skin, loss of vision and skin cancer after extended exposure.

### SUMMARY OF INVENTION

The present invention is directed to a germicidal lighting fixture that has controlled ventilation through a disinfecting chamber. The germicidal light fixture has a housing with a UV light engine contained therein that forms the disinfecting chamber. Air passed through the disinfecting chamber, thereby passing air in a vicinity of the UV light engine and expelled sanitized or disinfected air out from the disinfecting chamber. To control the air flow through the disinfecting chamber, the germicidal light fixture has one or more centrifugal fans, turbines, moving baffles or ribbons that guide air through the disinfecting chamber in a controlled manner. Also, to control residency times that volumes of air spend within the disinfecting chamber, stationary baffles or structures protrude from interior walls of the disinfecting chamber.

The germicidal light fixture preferably includes sensors for monitoring volumes of air that has passed through the disinfecting chamber and signals a percentage of a volume of room air and/or the number of times a volume or room air has pass through the disinfecting chamber. The germicidal light fixture also preferably includes a sensor for turning off the light UV light engine when the disinfecting chamber is opened or accessed.

In further embodiments of the invention the germicidal light fixture also include task lighting, ozone sensors and or occupancy sensors, such as described in the U.S. provisional patent application Ser. No. 103,743, filed on Aug. 20, 2020 and titled “GROW-LIGHT SYSTEMS WITH GERMICIDAL LIGHTING,” the U.S. provisional patent application Ser. No. 63/102,943, filed on Jul. 9, 2020 and titled “GERMICIDAL LIGHTING WITH REVERSE OCCUPANCY DETECTION,” and the U.S. provisional patent application Ser. No. 63/206,142, filed January 29, 201 and titled “FLUENCE DETECTION IN GERMICIDAL LIGHTING,” the contents of which are all hereby incorporated by reference.

### BRIEF DESCRIPTION OF THE DRAWINGS

FIG. 1 shows a schematic representation of a germicidal light fixture with controlled ventilation, in accordance with embodiments of the invention.

FIGS. 2A-D illustrate side views of a germicidal light fixture, such as the germicidal light fixture with fans, turbines, moving baffles or ribbons that guide air through the disinfecting chamber in a controlled manner and with stationary baffles, ribbons or structures that protrude from interior walls of a disinfecting chamber, in accordance with embodiments of the invention.

FIGS. 3A-D show germicidal light fixtures with form factors that include ventilation and an accessible disinfecting chamber, in accordance with the embodiments of the invention.

FIGS. 4A-E show schematic representations of wiring configuration for germicidal light fixtures, in accordance with the embodiments of the invention.

### DESCRIPTION OF THE INVENTION

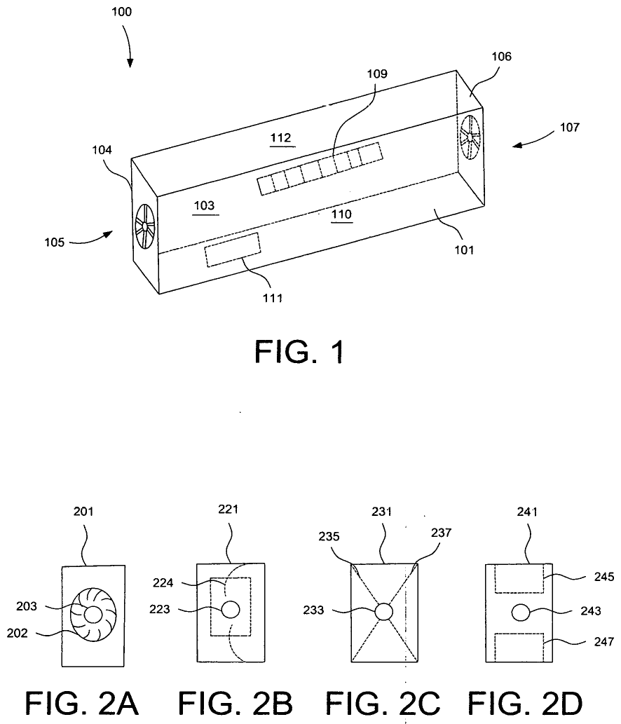

FIG. 1 shows a schematic representation of a germicidal light fixture 100 with controlled ventilation through a disinfecting chamber 103, as referred to as a germicidal chamber. The germicidal light fixture 103 has a housing 101 with housing walls 104, 106, 110 and 112 with a UV light engine 109 contained therein that forms the disinfecting chamber 103. Air passed through the disinfecting chamber 103, thereby passing air in a vicinity of the UV light engine 109 and expelled sanitized or disinfected air out from the disinfecting chamber 103. To control the air flow through the disinfecting chamber 103, the germicidal light fixture 100 has one or more centrifugal fans, turbines, moving baffles or ribbons 105 and 107 on opposed side walls 104 and 106 of the disinfecting chamber 103 that guide air through the disinfecting chamber 103 in a controlled manner. Also, to control residency times that volumes of air spend within the disinfecting chamber, stationary baffles, ribbons, tabs or other structures or structures protrude from interior walls 110 and 112 of the disinfecting chamber 103, such as described with reference to FIGS. 2A-D below.

It is undershoot that the germicidal light fixture 100 includes all of the necessary electronics, wiring and sensors 111 to power and control the germicidal light fixture 100 and allow the germicidal light fixture to operating in accordance with any number of operating protocols.

The germicidal light fixture preferably includes sensors for monitoring volumes of air that has passed through the disinfecting chamber and signals a percentage of a volume of room air and/or the number of times a volume or room air has pass through the disinfecting chamber. The germicidal light fixture also preferably includes a sensor for turning off the light UV light engine when the disinfecting chamber is opened or accessed.

FIGS. 2A-D illustrate side views of a germicidal light fixture, such as the germicidal light fixture 100 with fans, turbines, moving baffles or ribbons on one or more side walls, such as side walls 104 and 106, that guide air through the disinfecting chamber 103 in a controlled manner and with stationary baffles, ribbons or structures that protrude from interior walls 110 and 112 of a disinfecting chamber 103.

FIG. 2A shows a side view 201 of a germicidal light fixture, like the germicidal light fixture 100, with a fan 203 and ribbon structures 202 that interior walls 110 and 112 of the disinfecting chamber 103 extend through portions of the disinfecting chamber 103 of a germicidal light fixture 100.

FIG. 2B shows a side view 221 of a germicidal light fixture, like the germicidal light fixture 100, with a fan 223 and complex ribbon structures 2224 that protrude from interior walls 110 and 112 of the disinfecting chamber 103 extend through portions of the disinfecting chamber 103 of a germicidal light fixture 100.

FIG. 2C shows a side view 231 of a germicidal light fixture, like the germicidal light fixture 100, with a fan 233 and bar structures 2235 and 237 that protrude from interior walls 110 and 112 of the disinfecting chamber 103 and extend through portions of the disinfecting chamber 103 of a germicidal light fixture 100.

FIG. 2C shows a side view 231 of a germicidal light fixture, like the germicidal light fixture 100, with a fan 233 and tab structures 2235 and 237 that protrude from interior walls 110 and 112 of the disinfecting chamber 103 and extend into portions of the disinfecting chamber 103 of a germicidal light fixture 100.

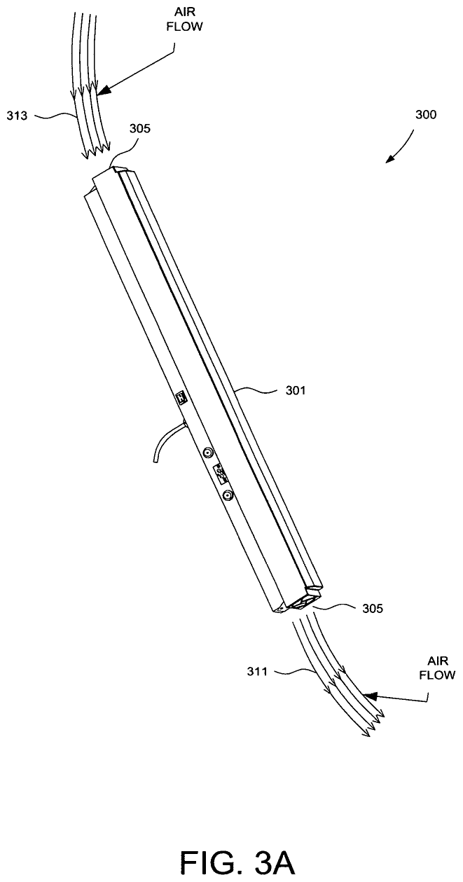

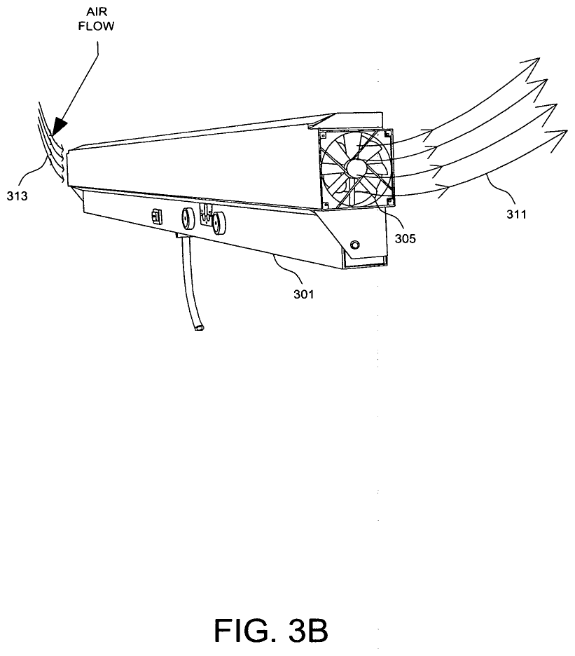

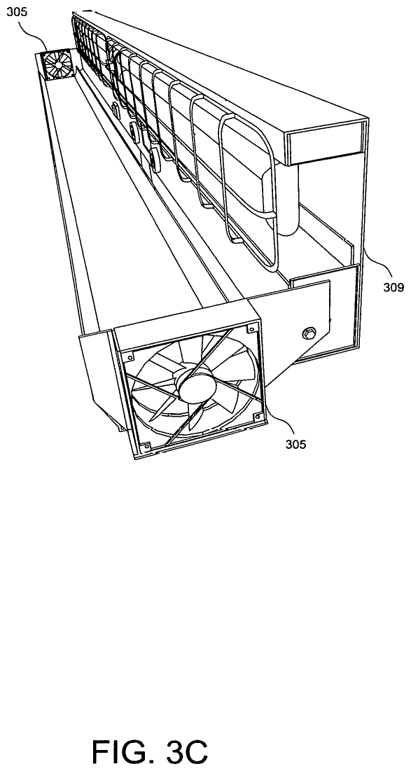

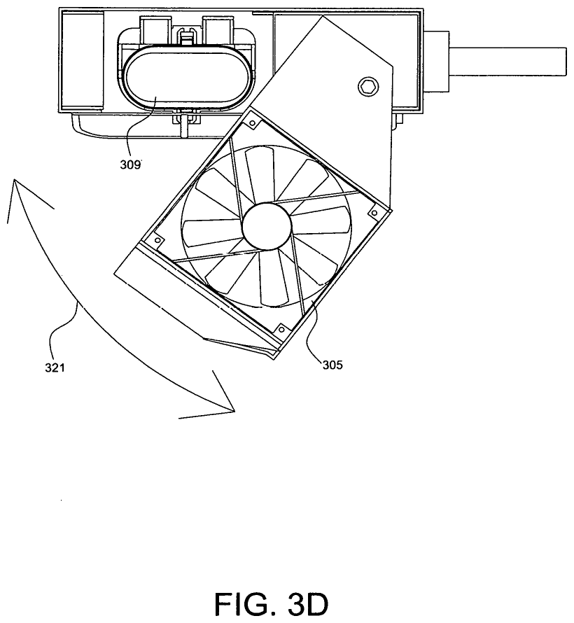

FIGS. 3A-D show views 300 of a germicidal light fixture 301. The germicidal light fixture 301 includes on or more ventilation fans 305 and 305′ that draws untreated air 313 into a germicidal chamber with a germicidal light engine therein and pushes exposed and disinfected air 311 out of the germicidal chamber, such as described above. The germicidal light fixture 310 can be a dual purpose light fixture that also provides task lighting and can include a hinge feature, such as shown in FIG. 3C-D that allows the germicidal light engine 309 in the germicidal chamber to be accessed, as indicated by the arrow 321, and serviced. Regardless of the form factor of the germicidal light fixture, the germicidal light fixture is preferably equipped with detection that shut the germicidal light engine when the germicidal chamber is accessed.

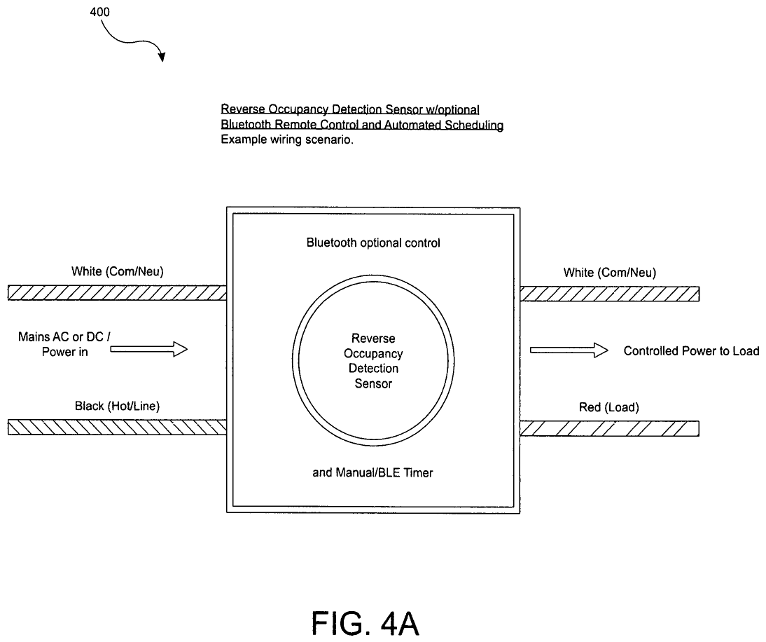

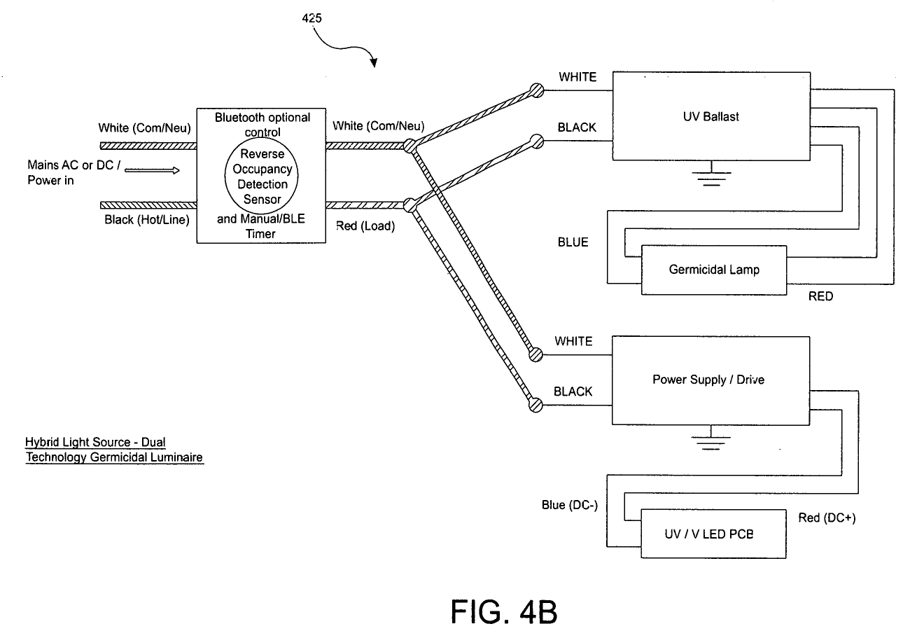

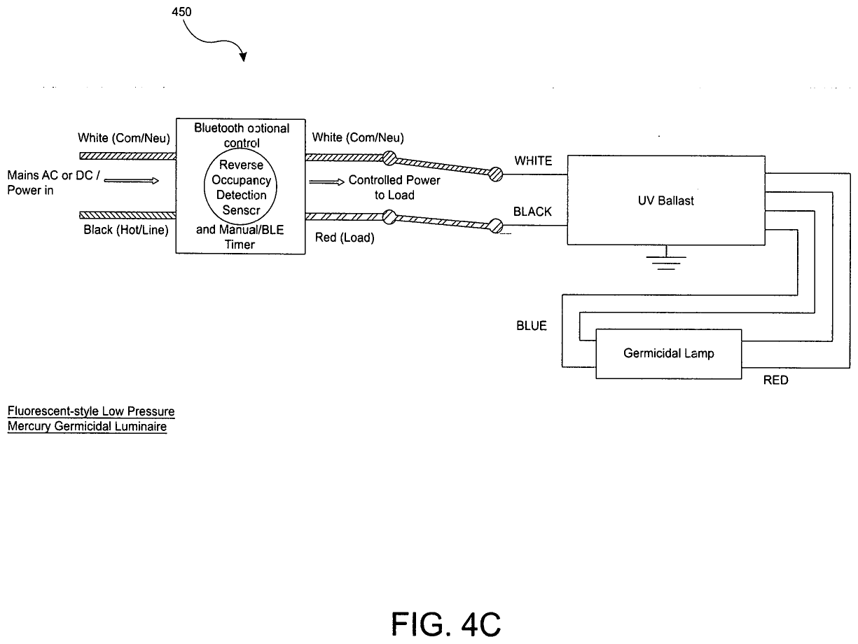

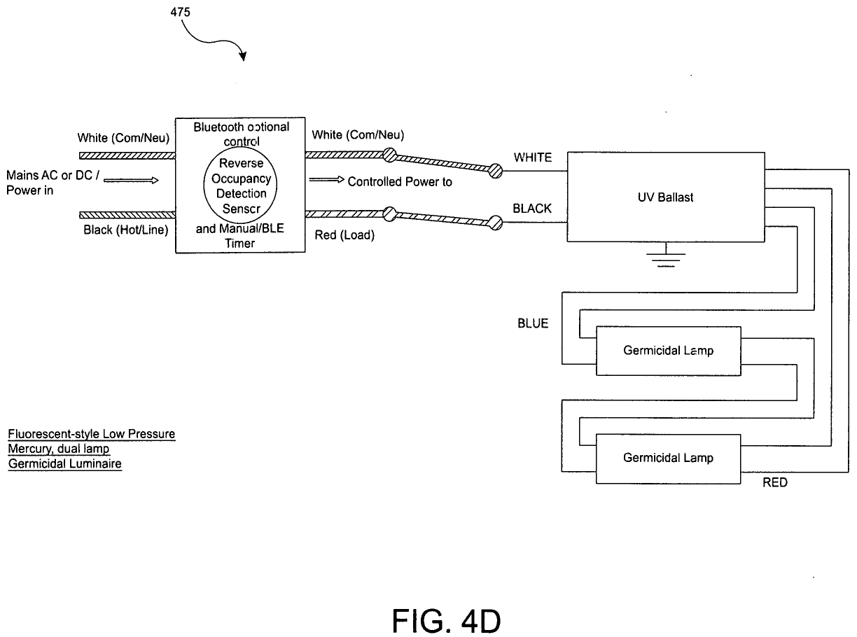

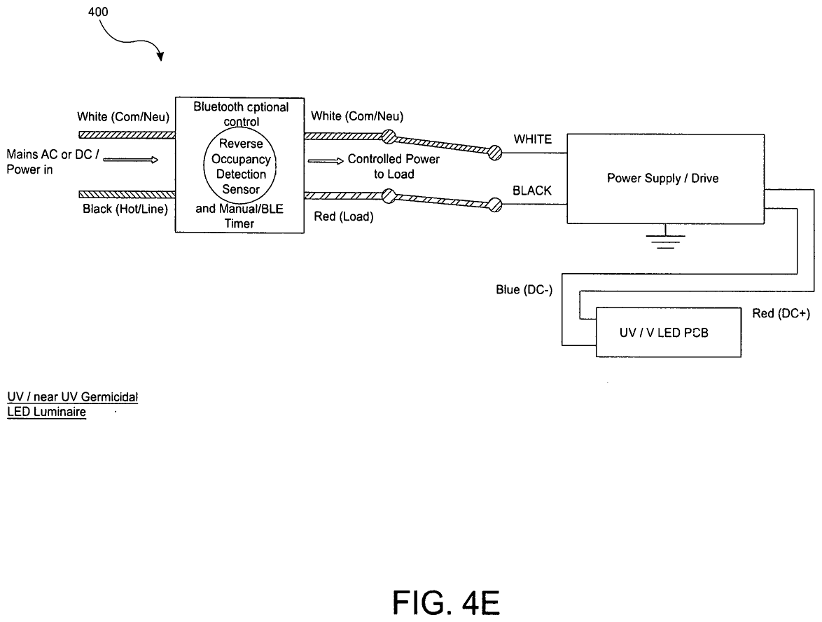

FIGS. 4A-E show schematic representations of wiring configuration for germicidal light fixtures, in accordance with the embodiments of the invention. FIG. 4A shows a schematic representation 400 of a reverse occupancy detection circuit with automatic scheduling for turning on germicidal lighting in the absence of occupants and/or turning on and off germicidal lighting according to a schedule. FIG. 4B shows a schematic representation 425 of wiring for operating a fixture with both task lighting and germicidal lighting. FIG. 4C shows a schematic representation 450 of wiring for powering a fluorescent-style low pressure mercury germicidal light engine. FIG. 4D shows a schematic representation 475 of wiring for powering multiple fluorescent-style low pressure mercury germicidal light engines. FIG. 4E shows a schematic representation 400′ of wiring for powering a UV/or near UV germicidal LED light engine.

What is claimed is:

1. A germicidal light fixture comprising:
a) a UVC lamp;
b) a housing that from a disinfecting chamber for housing the UVC lamp and having vents:
c) a fan for circulating air across surface of the UVC lamp through the vents; and
d) baffles, ribbons or tabs that protrude from interior walls of the disinfecting chamber.
